(19) United States
(12) Patent Application Publication
(10) Pub. No.: US 2023/0241276 A1
(43) Pub. Date: Aug. 3, 2023

(54) METHOD FOR VIRAL DISINFECTION OF AIR AND FOMITES IN A DEFINED SPACE

(71) Applicant: Dominique Mercier, Coulommiers (FR)

(72) Inventor: Dominique Mercier, Coulommiers (FR)

(21) Appl. No.: 17/995,281

(22) PCT Filed: Apr. 6, 2021
(86) PCT No.: PCT/EP2021/058858

§ 371 Date: Sep. 30, 2022

(30) Foreign Application Priority Data
Apr. 3, 2020 (FR) FR2003335

(51) Int. Cl. A61L 9/04; F24F 6/02

(52) U.S. Cl. CPC A61L 9/04; F24F 6/02; A61L 2209/212

(57) ABSTRACT

The invention relates to a method for viral disinfection of a defined space, having: diffusing, into the ambient air in the defined space, molecular ozone with an exposure dose CT that corresponds to the product of C × T, where C represents the concentration of molecular ozone and T represents the duration of treatment, said exposure dose CT being greater than 2 mg.min/m³, preferably substantially constant and equal to 5 mg.min/m³; and regulating the humidity level of the ozonized ambient air in the defined space.

### TECHNICAL FIELD

The present invention relates to the field of viral disinfection of defined spaces, in particular confined spaces, such as the internal volume of premises, decontamination airlocks (in particular before access to a confined space), goods or passenger vehicles, such as train carriages for example, or even smaller volumes of protection suit chambers or other antiviral protective clothing.

The invention thus aims to enable the effective disinfection of confined spaces and thus eliminate at least some of the disadvantages related to the techniques currently provided.

The present invention relates more precisely to a method for viral disinfection of the ambient air of such defined spaces and associated fomites. The present invention also provides different embodiments of systems capable of implementing such a method.

It is specified that the expression “associated fomites” here should be understood as surfaces, materials or objects contaminated with one or more viruses and likely to contaminate other objects, animals or people, thus playing a role in the spread of a contagious viral disease. For example, these fomites may consist of surfaces of tables, seats or other movable or stationary objects contained in the defined space under consideration.

Furthermore, it should be understood here, for the purposes of the invention, that a defined space corresponds either to a space likely to be substantially insulated in order to avoid uncontrolled leakage of the internal air it contains - in this case referred to as a confined or closed space, or to a defined volume in which moving air (continuous or batch) is exposed for a given time to one or more treatments, on the principle of a chamber for treating an air flow as for respiratory protection or treatment of air ducts for air distribution in a premise.

The present invention is thus configured to allow the inactivation of airborne viruses and/or, where applicable, deposited on fomites present in defined spaces as previously illustrated, as well as in the air and air-conditioning installations associated with them for the renewal of hygienic air and/or thermal comfort.

### BACKGROUND

It is known from the state of the art the possibility to carry out viral disinfection of fomites by the use of virucidal products, either by manual application (a), by air (b) or by the use of UVC germicidal radiations (c).

According to (a) and (b), virucidal products are applied by contact or spraying on the surfaces to be treated, the different materials making up the surfaces to be treated often requiring the use of different virucidal materials. However, indoor air often carries airborne viruses, so that, in the absence of appropriate treatment, it is likely to re-contaminate previously decontaminated surfaces.

According to (b), the virucidal agents may be gasses such as formaldehyde, chlorine dioxide or liquid gasses such as hydrogen peroxide.

According to (c), only the surfaces and the volume of air exposed directly to germicidal radiation at a limited distance are treated, in other words, the porous or fabric surfaces are not treated beyond their outer surface because they remain hidden from germicidal radiation.

Viruses encountered upon contamination of premises are likely to be present in aerosols (airborne) thus causing the indoor air to spread contamination.

According to (b), different techniques are currently provided for viral disinfection of fomites via ambient air. These methods for disinfection of fomites by air often have a limited impact on viral aerosols. Their uses and optimizations generally concern only decontamination of the fomites.

The virucidal agents used are either aerosolized liquids or gasses.

The implementation of these techniques requires expensive equipment and highly trained personnel.

Aerozolization of liquids is particularly challenging because its effectiveness depends on the size and homogeneity of the droplets.

For many years, different air disinfection techniques have been provided and claim a virucidal effect. The majority consists of equipment through which air circulates in contact with filters, ozone, photocatalysts, plasma, UVC radiation. These latter techniques can be combined in the same device. Only the air circulating in said equipment is supposed to have been treated, in the face of significant volumes of premises or fomites with large or complex surfaces, it is impossible to ensure that all ambient air has been treated while aerosolized viruses can continue to be found there, thus rendering this equipment ineffective in the face of large volumes of air to be treated and/or complex volumes or surfaces.

Residual virucidal activity beyond the equipment, beyond its treatment volume, is also observed with the use of ozone, in the state of the art. Furthermore, the efficiency of this gas is strongly affected, on the one hand, by its concentration in the air and, on the other hand, by the humidity rate of the air. In other words, the relative humidity of the ambient air affects the biocidal capacity of ozone when this gas is introduced into the ambient air to carry out its disinfection. This influence results from the formation of specific ions that are particularly oxidizing during ozone decomposition. Ozone is known to attack unsaturated bonds and form aldehydes, ketones or carbonyl compounds. Moreover, ozone can react very quickly with amino acids, proteins, protein functional groups and nucleic acids.

The reason for the importance of humidity in the ozone disinfection process is twofold. The first is physical. Higher humidity levels will enable the rehydration of dried pathogens, thus reducing their ozone faction resistance. Water also tends to form a thin layer around biological materials such as bacteria and viruses. When it comes to surface disinfection, this creates a wider surface contact between target pathogens and ozone. This is significant for the second reason why humidity is a decisive factor for the biocidal efficacy of ozone, as explained below.

Chemically, and in the presence of water, ozone depletion action can follow two distinct routes: either by direct action or by reaction intermediates called radical species. Because molecular ozone is highly reactive, it can also react with water and form transient radical species that have an extremely short but highly reactive lifespan. Similar to ozone, these species have a high oxidizing power and can in turn react with and inactivate harmful pathogens such as bacteria, viruses, fungi and yeasts.

State-of-the-art indoor air ozonation disinfection equipment is often provided for treatment in the presence of humans. According to the technical instructions for ozonation equipment intended for disinfection, the ozone rates implemented are a maximum of a few tenths of ppm/ml in the treated air at the outlet of the equipment, mainly with regard to the international and European standards which set the maximum ozone rate in the air. For example, in Europe, the limit value for the protection of human health is defined as a daily maximum of the 8-hour rolling average of 120 µg/m³, that is 0.061 ppm.

Several scientific studies have shown the need to reach several dozen ppm of ozone in indoor air to achieve partial inactivation of bacterial and fungal aerosols.

Depending on the pathogens encountered, their transmission modes via microdroplets and their ability to remain infectious when present in aerosols are poorly documented. However, the scientific community agrees that for a virus such as COVID 19, it can remain infectious for several hours in the air (aerosol) and for several days on inert surfaces.

Until now, the main mechanism by which ozone inactivates a virus was not well understood. However, the applicant’s research has led him to understand that ozone reacts with the virus by direct reaction with molecular ozone and by the radical species that form on decomposition thereof.

According to the object of the invention, the applicant therefore provides a method for inactivating viruses present in a space defined by the controlled use of molecular ozone injected into the ambient air of the defined space to be treated (that is, air in contact with the virus) and by regulating the relative humidity (that is, the humidity rate) of the ozonated ambient air in order to substantially increase the viral inactivation reactions for a given exposure dose to molecular ozone and therefore to decrease the necessary exposure dose compared to ozonation in air having a lower relative humidity. In addition, for low to medium humidity, it seems physically impossible to reach the required theoretical exposure doses for many pathogenic viruses.

Ozone is known to attack unsaturated bonds and form aldehydes, ketones or carbonyl compounds. Moreover, ozone can react very quickly with amino acids, proteins, protein functional groups and nucleic acids. Therefore, ozone would affect the protein structure of the viral capside or the nucleic acids of the virus.

### SUMMARY

More specifically, one object of the invention is a viral disinfection method for a defined space, comprising:
Molecularly rehydrating ambient air in the defined space; and
Diffusing, into the ambient air in the defined space, in particular confined space, molecular ozone with a CT exposure dose, corresponding to product C × T where C represents the concentration of molecular ozone and T represents the treatment time, preferably greater than 2 mg.min/m³ which is substantially constant and equal to 5 mg.min/m³.

Molecularly rehydrating ambient air in the defined space is performed by:

Generating negative air ions and contacting the ambient air to be treated with the negative air ions generated, and/or by:

Forcedly humidifying the ambient air in the defined space and regulating the humidity of the ozonated ambient air in the defined space.

According to one embodiment, regulating the humidity rate of the ambient air in the defined space is configured so that said humidity rate in the ambient air in the defined space is maintained above 70%, preferably substantially constant and equal to 85%, for an ambient air temperature substantially equal to 25° C.

According to one embodiment, regulating the humidity rate in the ambient air in the defined space comprises humidifying said ambient air in the defined space, so as to increase the concentration of decomposition products of molecular ozone, especially hydroxyl ions and free radicals, in the ambient air of the defined space.

According to one embodiment, the implementation of the method being carried out for a total duration;
Diffusing molecular ozone is carried out for a first duration less than the total duration;
Humidifying the ambient air in the defined space is carried out after the first duration has elapsed and for a duration ending at the latest with the total duration.

According to one embodiment, the method comprises the CT exposure dose greater than 2 mg.min/m³ for the treatment of ambient air in the defined space only.

According to one embodiment, the exposure dose is greater than 200 mg.min/m³ for the treatment of fomites in the defined space.

According to one embodiment, the method further comprises a final step of depleting residual molecular ozone in the ambient air of the treated defined space.

According to one embodiment, the method comprises a prior step of confining the defined space to make it a confined space.

The invention also aims at a system for viral disinfection of a defined space comprising an ozonizer and a humidifier configured to implement the method briefly described above.

According to one embodiment, the ozonizer comprises an electric arc generation device to create a corona effect or an UV-C type ultraviolet radiation generation device having a wavelength between 160 nm and 220 nm.

Advantageously, the humidifier comprises a device for adiabatic evaporation by vaporization or ultrasound emission or spraying.

Furthermore. It is known to the applicant that air humidification as described in the invention has significant technical and therefore financial restrictions. Besides, the presence of humidity in the air subjected to ionization leads to the formation of oxidizing compounds for metals such as nitric acid from the nitrogen of the air, hence the need identified by the applicant to perform humidification of the air after the ozonation sequence.

According to an alternative of the invention, the humidification sequence can be advantageously replaced by performing a negative ionization sequence of the air to be treated prior to its ozonation.

### BRIEF DESCRIPTION OF THE DRAWINGS

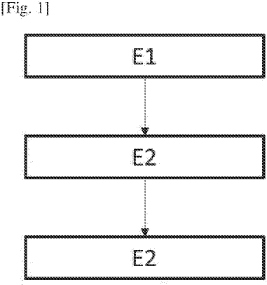

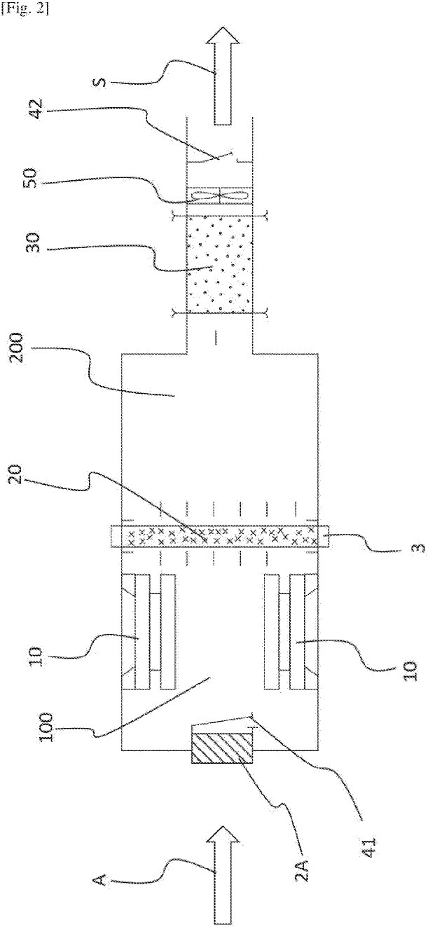

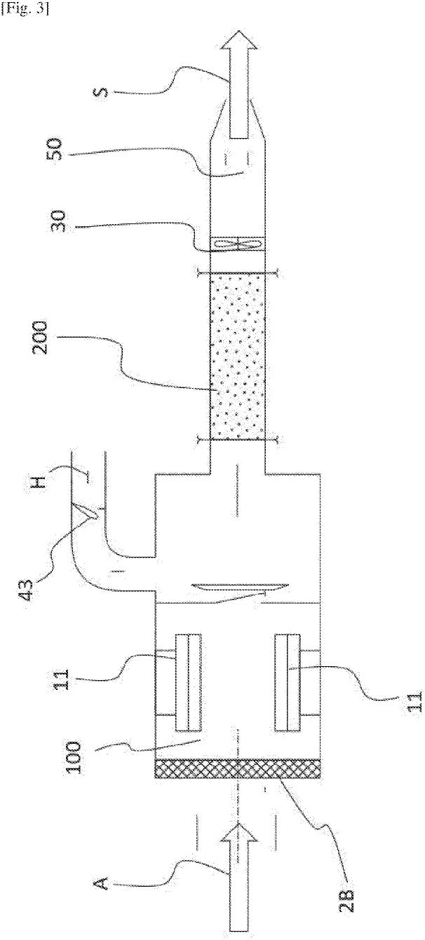

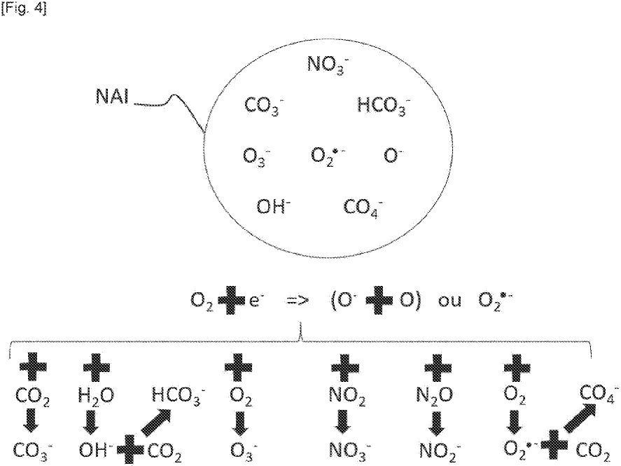

The invention will be better understood upon reading the following description, given as an example, and by referring to the following figures, given as non-limiting examples, in which identical references are given to similar objects.

[FIG. 1]: [FIG. 1] is a block diagram representing an embodiment of the method according to the invention;

[FIG. 2]: [FIG. 2] is a schematic representation of an example of a disinfection chamber implementing the invention;

[FIG. 3]: [FIG. 3] is a schematic representation of another example of a disinfection chamber implementing the invention;

[FIG. 4]: [FIG. 4] is a schematic representation of the decomposition and recomposition of negative air ions.

It should be noted that the figures disclose the invention in detail in order to implement the invention, said figures can of course be used to better define the invention if necessary.

### DETAILED DESCRIPTION

The invention is set forth mainly for the purpose of enabling viral disinfection of air and associated fomites in a defined space. However, other applications of the method according to the invention may be contemplated, especially a mobile viral disinfection unit for the supply air of devices such as breathing masks or tight suits used by interveners in virus-contaminated atmospheres.

As indicated above, the present invention relates to a method for viral disinfection of air and associated fomites in a defined space. It is therefore necessary to define the necessary ozone doses and the necessary humidity rate to obtain the contemplated viral infection. To do this, several definitions should be provided.

According to the applicant’s research, the ozone dose to which an airborne virus has been exposed is defined as the product of the concentration of ozone on the virus and contact time (TC). The survival fraction (FS, unitless) is a ratio that represents the concentration of virus after ozone exposure: FS = Ns/No = c^{-KTC} [055] with:
Ns is the concentration of surviving airborne viruses in the ambient air in the defined space after exposure to molecular ozone;
No is the initial concentration of airborne viruses in the ambient air in the defined space to be treated (before exposure to ozone);
C is the concentration of molecular ozone in the ambient air in the defined space (mg/m³);
T is the ozone/air contact time in minutes;
K is the susceptibility of the virus to ozone (m³/mg.min).

The exposure dose of molecular ozone, here determined as necessary, said exposure dose also being designated CT (product C × T where C is the concentration of molecular ozone and T is the treatment time or, in other words, the contact time between molecular ozone and the ambient air to be treated), is greater than 2 mg.min/m³ of ozonated air (that is, 1.02 ppm) and preferably substantially equal to 5 mg.min³ of ozonated air (that is, 2.55 ppm) for removal of the majority of known viruses at 99% under a relative humidity greater than 70%, preferably equal to 85%, and at 25° C. ambient temperature.

With reference to [FIG. 1], the method according to the invention thus comprises a step E1 of diffusion of molecular ozone into the ambient air of the defined space to be treated and then a step of forced humidification, that is, injection of water under nebulized ozone for example, and regulation of the humidity rate in the ozonated ambient air in the defined space.

For example, the total treatment duration may be between 6 minutes and 8 minutes, of which a first duration of between 1 minute and 2 minutes of concentrated molecular ozone injection, and a second duration of humidification of the ozonated ambient air, for a duration between 5 minutes and 6 minutes.

Preferably, there is then provided a step E3 of depletion of the residual molecular ozone in the ambient air of the treated defined space.

According to the applicant’s research, due to the complexity of the surfaces, materials and surface deposits encountered, the fomite(s), which are treated by fallout, have to be treated at a much higher ozone exposure dose or CT than for viral aerosols present in the ambient air of the defined space, in particular in the order of 100 times the exposure dose required for the treatment of ambient air only, subject to a rapid increase in the relative humidity of the ozonated air. For the treatment of the ambient air in the defined space and the fomites it contains, the following is recommended:
Step E1: Molecular ozone is diffused into the defined space so that ambient air in the defined space is exposed to molecular ozone with a CT value (or exposure dose) for treatment of fomites greater than 200 mg.min/m of ozonated air (approximately 102 ppm) and preferably also substantially equal to 500 mg.min/m³ of ozonated air (approximately 255 ppm);
Step E2: especially immediately following the exposure time determined by the criterion relating to the maintenance of the exposure dose (or CT) determined above, humidification of the ambient air in the defined space is carried out to obtain a rapid increase in the relative hygrometry of the ozonated air; in particular, the humidity rate in the ozonated ambient air in the defined space is made and maintained above 70%, preferably equal to 90%, by means of a humidifier, for a time greater than 1 minute and preferably substantially greater than 5 minutes, or even substantially equal to 6 minutes.

In this way, the majority of known viruses are eliminated at more than 99% at 25° C. ambient temperature.

According to the applicant’s research, it therefore appears that the exposure dose required by molecular ozone for the treatment of fomites is 100 times higher than the exposure dose required to inactivate airborne viruses. This is due to the complexity of the surfaces (grooves, cracks, porosity, etc.), the substrate materials and the virus support matrix.

If the defined space is not initially confined, a step of confining the defined space is preferably carried out before implementing the step E1 of the method, in order to make this space confined.

Such a confined space is defined as a totally or partially confined space, with the following characteristics: i) the means of access, both to the outside and the inside, are restricted; in particular, a confined space is made substantially airtight; ii) optionally, this space is not designed in advance or configured to be occupied by personnel working inside; iii) sometimes, when entering these spaces, operators may be exposed to a significant number of risks that have to be controlled.

With reference to FIGS. 2 and 3, two alternative embodiments of a system capable of implementing the method described above, wherein ambient air to be treated is exposed to molecular ozone in an ozonation chamber 100, then the ozonated ambient air is humidified so as to ensure sufficient humidity rate in a humidification chamber 200.

In the example in [FIG. 2], the generation of molecular ozone in the ozonation chamber 100 is performed by an especially mobile ozonizer 10, also known as an ozone generator, consisting mainly of UV-C lamps emitting light with a wavelength of about 185 nm. UV-C lamps generating molecular ozone emit UV-C type ultraviolet radiation with a wavelength between 160 nanometers and 220 nanometers, generally substantially equal to 185 nanometers.

Ozone is created when the dioxygen C molecules are separated by radiation, the dioxygen molecules split into two and then form O₃, that is, molecular ozone molecules.

Said UV-C lamp(s) are arranged in an ambient air stream to be treated, in particular generated by a fan (not represented).

In the example in [FIG. 3], the generation of molecular ozone in the ozonation chamber 100 is carried out by another type of ozonizer 11, namely a corona-effect ozone generator. Such an ozonizer 11 – a corona-effect ozone generator – generates an electric arc that causes dioxygen 02 to be recomposed to ozone 03. As in the other example, the corona-effect ozonizer 11 is placed in an ozonation chamber 100, the volume of which is filled with air drawn into the defined space to be treated, said air being ozonated into said ozonation chamber 100 before being discharged into the ambient air of the defined space. However, it is noted that the preferred embodiment for the generation of molecular ozone is the use of UV-C lamp(s) because corona-effect ozone generators have the disadvantage of requiring very dry air to operate optimally. Without dry air, corona-effect ozone generators have an ozone production that reduces over time and their lifespan becomes very short because the metal elements that create the electrical field easily oxidize in humid ambient air.

An UV-C lamp, on the other hand, does not need any dry air and does not oxidize, making it more adapted for use in preferably humid ambient air, as explained above.

The ambient air to be treated A is preferably drawn into the defined space to be treated by means of a fan leading into an ozonation chamber in which the UV-C lamp(s) is (are) arranged, especially after passing through a filter 2A, 2B, which may be a washable dust filter. A check valve 41 can be provided to prevent backflow of air into the ozonation chamber 100.

After sufficient contact time between the molecular ozone diffused by the ozonizer 10, 11 and the ambient air to be treated in the ozonation chamber 100, the ozonated ambient air is directed out of the ozonation chamber into the defined space to be treated.

According to one embodiment, the operation of the ozonizer 10, 11 is controlled and driven by a probe for measuring the molecular ozone rate, arranged in the defined space to be treated. Said probe has, for example, a means of cutting off the supply of the ozonizer 10 when the expected molecular ozone rate (that is, when the ozone concentration in the ambient air of the defined area to be treated ensures, taking account of the treatment time, an exposure dose (or CT) above the predefined threshold) in said defined area is reached.

At the end of the ozonation step E1, there is the forced humidification and humidity regulation step, in other words the humidification of the ozonated ambient air. Humidification of the air consists in particular in injecting water in the form of a wet mist, in other words in the form of microdroplets, so that it is absorbed by the ozonated ambient air, so as to increase the humidity rate of said ozonated ambient air. The finer the micro droplets, the better the absorption. Air humidification techniques are mainly by evaporation (adiabatic), vaporization, ultrasounds or spraying.

According to one embodiment not represented, humidification of the ozonated ambient air is carried out in the space defined by a humidifier consisting of a mobile electric air mister.

According to one embodiment, the start-up of the humidifier is preferably performed automatically, at the time of shutdown of the ozonizer, in other words after the end of the step E1 of ozonation of the ambient air of the defined space.

According to one embodiment, the operating duration of the humidifier, in particular the air mister, is driven via a relative humidity probe disposed in the defined space to be treated.

With reference to FIGS. 2 and 3. humidification of the ozonated ambient air is carried out in a humidification chamber 200 arranged at the outlet of the ozonation chamber 100. For example, with reference to [FIG. 2], a humidifier 20 is provided at the inlet of the humidification chamber 200 next to the ozonation chamber 100. The humidifier 20 is then, for example, an adiabatic humidification cassette containing foam or felt humidified via a water reservoir 3.

In the example in [FIG. 3], humid air H, for example from recirculating exhaled air of the user, is injected into the humidification chamber 200 via a pipe with a check valve 43.

According to one embodiment, at the end of the treatment phase under controlled humidity, corresponding to the step, during which a humidity rate in the ambient air to be treated above the predefined threshold is ensured, an ozone depleter 30, particularly by catalysis, is operated in the treated defined space. Especially, the shutdown of the ozone depleter 30 is driven via the probe for controlling the ozone rate when it detects a concentration of molecular ozone in the treated ambient air of less than 1 ppm.

In FIGS. 2 and 3, the ozone depleter 30 is schematically represented as a catalyst filter. The person skilled in the art has a whole panel of known catalysts available to perform this function. In particular, reference may be made to the PhD document “Synthèse et caractérisation de nouveaux catalyseurs hétérogènes pour la dépollution de l′air”, presented and defended by Benjamin FAURE on 9 Dec. 2014, at the Toulouse 3 Paul Sabatier University.

Advantageously, according to a preferred embodiment, the ozonation (ozonizer 10, 11), humidification (humidifier 20) and ozone depletion (ozone depleter 30) devices are driven by means of a control module 50 connected to a probe for measuring the ozone content in the ambient air of the defined space to be treated and to a relative humidity probe, configured to measure the humidity rate of the ambient air, in particular ozonated ambient air, in the defined space, said probes being arranged in the defined space to be treated so that the viral disinfection method according to the invention is likely to be implemented without human presence, in particular in the defined space to be treated. A check valve 42 can be provided at the system outlet and the outgoing air S is returned to the ambient air in the defined space.

For this purpose, the system comprises a probe to measure the ozone concentration in ambient air. The ambient air of the defined space is considered to be breathable when, after implementation of the viral disinfection method, the concentration of molecular ozone in the ambient air is reduced to below 1 ppm.

In the event that no ozone depleter configured to reduce the residual molecular ozone in the ambient air of the treated defined space is provided, it is reminded that molecular ozone naturally degrades, its half-life being reduced. As a reminder, the half-life of molecular ozone is thus in the order of a few days at temperatures between -25° C. and +20° C. (around 3 days at 20° C.), said half-life reducing with the increase in temperature. Humidity also reduces the half-life of ozone.

As a result, the concentration of molecular ozone in the ambient air of the defined space will naturally fall below the above threshold after a few hours

Other devices (not represented and briefly described below) are provided to enable viral disinfection of air and associated fomites in particular defined spaces.

In short, the present invention relates to a method for viral disinfection of air and associated fomites in a defined space. More specifically, the present invention relates to a method for viral disinfection of ambient air and associated fomites in a defined space comprising an ozonation step immediately followed by a step of rapid humidification of the ozonated ambient air in order to increase the formation of hydroxyl ions and radical species during the decomposition of molecular ozone in contact with water vapor and thus, on the one hand, to optimize the destruction of viruses contained in the ambient air to be treated and, on the other hand, to also reach certain encapsulated viruses or viruses embedded in organic matrices while significantly reducing the necessary dose of exposure to molecular ozone compared to lower humidity. This decrease in the necessary exposure dose (or CT) thus makes molecular ozonation compatible with the use and/or technically limiting restrictions.

Therefore, the present invention allows a direct action of the ozone molecule O₃ and then the action of hydroxyl ions and free radicals, decomposition products of molecular ozone, on airborne viruses and fomites.

Indeed, molecular ozone primarily acts on the capsides (a kind of protective protein shell) of airborne viruses in the ambient air of the defined space to be treated, but it also acts on the nucleic acids of these viruses. Hydroxyl ions and free radicals, resulting from the decomposition of ozone molecule and the presence of which is enhanced in the presence of humidity, act on the content of the capside of the virus, namely at least one nucleic acid generally stabilized by basic nucleoproteins. The ambient air to be treated is thus treated by the association of molecular ozone diffusion, with a dose of exposure maintained above a predefined threshold, and regulation of sufficient humidity rate.

It is further specified that the present invention is not limited to the examples described above and is susceptible of many variations accessible to the person skilled in the art.

### Applications Contemplated

Several uses of the invention are contemplated, without the list given below being construed restrictively.

Firstly, for viral disinfection of premises or cars (individual vehicles, busses, trains, planes, etc.), it is provided to carry out the initial step of confining the defined space to be treated, so as to make it hermetic and thus allow greater treatment efficiency. Then, the system as defined above, previously installed in said space, is put into operation. It is noted that the ozonizer and the humidifier can be two separate machines or two devices encapsulated in a same machine.

It is also provided to implement the invention to disinfect a disinfection airlock for access to an infected confined space. In this case, persons configured to enter this infected confined space are equipped with a breathing mask when they are in the disinfection airlock.

The invention is also configured to be used to make individual and portable self-contained units. Such self-contained units are, for example, integrated into a protection suit for people working in infected environments. In such a self-contained unit, molecular ozone is produced in the ozonation chamber, by corona effect or UV-C lamp, according to an incoming air flow. The ozone production is adjusted according to the incoming air flow with a concentration adapted to a treatment time of 10 to 20 seconds, for example, in order to ensure the required exposure dose. The ozonated air is humidified by any humidifier, especially capable of taking advantage of the humidity present in the air exhaled by the person equipped with the protection suit, then the treated air passes through a catalyst filter to deplete residual molecular ozone so as to form an air flow configured to ventilate said person.

It is also provided to implement the invention to disinfect air or air-conditioning ducts supplying premises with disinfected air.

### Alternative Embodiment: Corona Ions

Air ions, hereinafter referred to as NAI (negative air ion(s), have electrically charged molecules or atoms in the atmosphere. An air ion is formed when a molecule or gas atom receives sufficient energy to eject an electron. Negative ions in NAI air are those that gain an electron, while positive ions in air lose an electron.

Artificial corona discharge is an effective way to generate NAIs. When a high negative voltage is applied to a conductor or electrode and the electrical field generated is high enough, corona discharge occurs. If a charged conductor/electrode has a needle type with a sharp tip, the electrical field around the tip will be much higher than other parts and air near the electrode may become ionized and NAIs are generated. The intensity of the corona discharge depends on the shape and size of the conductors as well as the applied voltage. An irregular conductor, especially with a sharp tip, results in more crown than a smooth conductor and large diameter conductors produce a smaller crown than small diameter conductors. The higher the applied voltage, the more NAIs are generated. The closer the distance to the corona point, the higher the concentration of NAIs, as the continuous generation of NAIs by corona discharge is related to a chain reaction process called electron avalanche. Corona discharge produces air ions and ozone. Ozone production can be limited and lowered below the detection threshold by adjusting discharge parameters.

In general, generated NAIs are not stable and will be progressively degraded. In the presence of humidity (even low) and water droplets even if they are condensed after partial evaporation, NAIs released into the ambient air easily combine with water molecules and thus form clusters of negative ions of larger size and longer lifespan, NAIs consist of several negatively charged molecules and these negative ions combine with several or up to 20 or 30 water molecules and form clusters of negative ions such as CO₃ — (H₂O)ₙ, O — (H₂O)ₙ and O₃ — (H₂O)ₙ. The viral loads making up the aerosols, especially in the form of droplets initially condensed by evaporation, are then held in these clusters.

With reference to [FIG. 4], NAIs can change from one NAI to another NAI. For example, NAIO- is formed when an oxygen molecule O2 obtains an electron. NAIO- can contribute to the formation of secondary NAIs by collision-assisted electron attachment processes when other molecules exist in the same space. As a result, other NAIs are generated such as O2 —, CO4 —, CO3 —, OH —, HCO3 —, O3 —, NO3 — and NO2 — the time change of NAI is related to the composition of ambient air. NAIs change continuously when they collide with molecules in the air. Thus, NAIs are dynamic in their composition, which depends on ionization potential and electronic affinity, proton affinity, dipolar moment and polarizability as well as the reactivity of the molecule.

It is useful to specify that, for the purposes of the invention, the injection of NAI into the ambient air prior to its ozonation has two advantages:
They promote the formation of molecular clusters of water that collect viral loads;
They consist partly of oxidizing ions and free radicals.

According to the object of this alternative of the invention, the aggregation and hydration of aerosolized viral particles in clusters (H₂O)ₙ formed by the action of NAIs replace favorably or complement forced humidification of ambient air as set forth in the first embodiment of the invention, in step E2, described above.

In other words, to rehydrate the viral particles in the ambient air, the alternative embodiment provides that the NAI ions injected into the air cause the formation of aggregates of water molecules and hence the molecular hydration of the aerosolized viral loads, in particular by the rehydration of the viral droplets condensed by partial evaporation.

Thus, the method according to the invention causes in particular the oxidation of viral loads by the addition of ions and oxidizing radicals within aerosolized H₂O molecular clusters, for which it causes the formation.

This embodiment replaces or supplements the first embodiment described, according to which forced humidification of the ambient air to be treated is carried out, within the defined volume, for example by means of water injection, in particular nebulized injection water, which results in rehydration of the ambient air corresponding to a diffusion of clusters of water molecules.

In addition, the presence of oxidizing ions and oxidizing radicals participates upstream and then in the presence of ozone, in the inactivation of viral loads. In this respect, it is specified that the step of ionizing (generation and injection of negative air ions) and mixing these ions with the ambient air can be used in the presence of humans and can therefore be maintained preventively between two phases of ozonation treatment (with or without humidification) which could only be implemented here if contamination is suspected (curative treatment).

According to this alternative of the invention, the ambient air to be treated is brought in by a mechanical means in contact with the NAI generator corona device, the air loaded with NAIs is then diffused by any means into the ambient air.

According to experiments conducted by the inventor, the speed of passage of the air to be treated in contact with the NAI generator should preferably be less than 1 m/s and still preferably around 0.5 m/s

For greater efficiency, the production of NAIs in the treated air to achieve the expected effects has to be greater than 100,000 ions/cm³ and preferably in the order of 450,000 ions/cm³ of air flow passing in contact with the generator. The NAI concentration in ambient air should be greater than 1000 ions/cm³ and preferably around 5000 ions/cm³.

Alternatively, fibers, woven materials or profiles made of plastic, preferably PTFE, are arranged in the air flow upstream of the NAI generator in order to positively charge the water molecules in the air to be treated with a triboelectric effect. This arrangement promotes the speed of attraction between the latter and the NAIs.

1-13. (canceled)

14. A method for viral disinfection of a defined space, comprising:
- molecularly rehydrating ambient air in the defined space; and
- diffusing, into the ambient air in the confined space, molecular ozone with an exposure dose CT, corresponding to the product C × T where C represents the concentration of molecular ozone and T represents the treatment time, preferably greater than 2 mg/min/m³ which is substantially constant and equal to 5 mg/min/m³.

15. The method according to claim 14, wherein the molecular rehydration of the ambient air in the defined space comprises generating negative air ions and contacting the ambient air to be treated with the negative air ions generated.

16. The method according to claim 14, wherein the molecular rehydration of the ambient air in the defined space comprises forcedly humidifying ambient air in the defined space and regulating the humidity rate of ozonated ambient air in the defined space.

17. The method according to claim 16, wherein regulating the humidity rate of the ambient air in the defined space is configured so that said humidity rate of ambient air in the defined space is maintained greater than 70%, preferably substantially constant and equal to 85%, for an ambient air temperature substantially equal to 25° C.

18. The method according to claim 16, wherein regulating the humidity rate in the ambient air in the defined space comprises humidifying said ambient air in the defined space, so as to increase the concentration of molecular ozone decomposition products, comprising hydroxyl ions and free radicals, in the ambient air of the defined space.

19. The method according to claim 18, wherein the implementation of the method being carried out for a total duration:
- the diffusion of molecular ozone is carried out for a first duration less than the total duration;
- Forced humidification of the ambient air in the defined space is carried out after the first duration has elapsed and for a second duration ending at the latest with the total duration.

20. The method according to claim 14, wherein the exposure dose is greater than 2 mg.min/m³ for the treatment of ambient air in the defined space only.

21. The method according to claim 14, wherein the exposure dose is greater than 200 mg.min/m³ for the treatment of fomites in the defined space.

22. The method according to claim 14, further comprising a final step of depleting residual molecular ozone in the ambient air of the treated defined space.

23. The method according to claim 14, comprising a prior step of confining the defined space to make it a confined space.

24. A system for viral disinfection of a defined space, comprising an ozonizer and a humidifier configured to implement the method according to claim 17.

25. The system according to claim 24, wherein the ozonizer comprises an electric arc generation device to create a corona effect or an UV-C type ultraviolet generation device having a wavelength between 160 nm and 220 nm.

26. The system according to claim 24, wherein the humidifier comprises an adiabatic evaporation device by vaporization or ultrasound emission or spraying.
